(19) United States
(12) Patent Application Publication
(10) Pub. No.: US 2023/0115396 A1
(43) Pub. Date: Apr. 13, 2023

(54) COMPUTER-READABLE RECORDING MEDIUM STORING COMPOUND SUBSTITUTION PROGRAM, METHOD, AND DEVICE

(71) Applicant: FUJITSU LIMITED, Kanagawa (JP)

(72) Inventor: Kazunari TANAKA, Kawasaki (JP)

(73) Assignee: FUJITSU LIMITED, Kanagawa (JP)

(21) Appl. No.: 18/065,443

(22) Filed: Dec. 13, 2022

Related U.S. Application Data
(63) Continuation of application No. PCT/JP2020/029451, filed on Jul. 31, 2020.

(51) Int. Cl. G16C 20/30; G16C 20/40

(52) U.S. Cl. CPC G16C 20/30; G16C 20/40

(57) ABSTRACT

A non-transitory computer-readable recording medium stores a compound substitution program for causing a computer to execute processing including: specifying a first partial structure included in a first compound; referring to information that indicates a relationship between a plurality of partial structures and selecting a second partial structure related to the first partial structure; determining whether or not a score calculated based on an appearance status of a group that includes the first partial structure and the second partial structure in a plurality of pieces of text data is equal to or more than a threshold; and generating information that indicates a second compound obtained by substituting the first partial structure of the first compound with the second partial structure, in a case where it is determined that the score is equal to or more than the threshold.

### CROSS-REFERENCE TO RELATED APPLICATION

This application is a continuation application of International Application PCT/JP2020/029451 filed on Jul. 31, 2020 and designated the U.S., the entire contents of which are incorporated herein by reference.

### FIELD

The embodiment discussed here is related to a compound substitution technology.

### BACKGROUND

In the field of chemistry, there is a case where documents such as patent publications or papers are searched by specifying a compound name as a key. At this time, it is useful to obtain documents regarding not only a compound indicated by the compound name specified as a key and but also compounds having similar structures with the compound. For this, traditionally, a technique has been proposed for specifying a compound that has a similar structure to the compound indicated by the compound name specified as a key and searching for a document regarding the specified compound.

Japanese Laid-open Patent Publication No. 11-175552 and Japanese Laid-open Patent Publication No. 2007-153767 are disclosed as related art.

### SUMMARY

According to an aspect of the embodiments, a non-transitory computer-readable recording medium stores a compound substitution program for causing a computer to execute processing including: specifying a first partial structure included in a first compound; referring to information that indicates a relationship between a plurality of partial structures and selecting a second partial structure related to the first partial structure; determining whether or not a score calculated based on an appearance status of a group that includes the first partial structure and the second partial structure in a plurality of pieces of text data is equal to or more than a threshold; and generating information that indicates a second compound obtained by substituting the first partial structure of the first compound with the second partial structure, in a case where it is determined that the score is equal to or more than the threshold.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating a configuration example of a compound substitution device;

FIG. 2 is a diagram illustrating an example of a data structure of score information;

FIG. 3 is a diagram illustrating an example of a data structure of a componentization rule;

FIG. 4 is a diagram illustrating an example of a knowledge graph;

FIG. 5 is a diagram for explaining processing of obtaining compounds having similar structures;

FIG. 6 is a flowchart illustrating a flow of processing of calculating a score;

FIG. 7 is a flowchart illustrating a flow of processing of obtaining similar compounds; and

FIG. 8 is a diagram for explaining a hardware configuration example.

### DESCRIPTION OF EMBODIMENTS

However, the related art has a problem in that it may be difficult to specify compounds having similar properties.

For example, according to the related art, it is possible to obtain a second compound that has a structure similar to a first compound by substituting a partial structure of the first compound with a partial structure corresponding to a subordinate concept belonging to the same superordinate concept. For example, a similar compound can be obtained by substituting propyl of “2,2-bis(4-hydroxyphenyl)propane” (bisphenol A) with another alkyl group.

Here, it can be said that a compound obtained by substituting propyl of bisphenol A with butyl is similar to the original bisphenol A in terms of structure and property. On the other hand, it can be said that a compound obtained by substituting propyl of bisphenol A with pentyl is similar to the original bisphenol A in terms of structure, because the compound and the bisphenol A have partial structures of the same alkyl group. However, since a chain becomes longer, there is a case where it cannot be said that properties are similar to each other.

In one aspect, an object is to specify compounds having similar properties.

Hereinafter, an embodiment of a compound substitution program, method, and device will be described in detail with reference to the drawings. Note that the embodiment does not limit the present invention. Furthermore, the individual embodiments may be appropriately combined within a range without inconsistency.

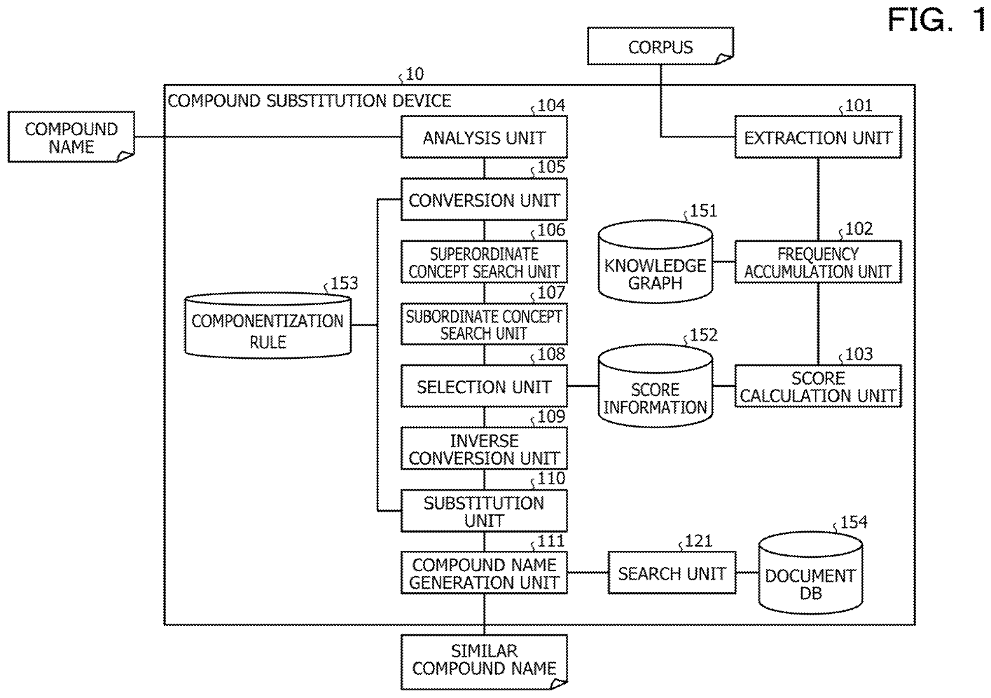

A configuration of the compound substitution device according to the embodiment will be described with reference to FIG. 1. FIG. 1 is a diagram illustrating a configuration example of a compound substitution device. As illustrated in FIG. 1, a compound name and a corpus are input to a compound substitution device 10. Furthermore, the compound substitution device 10 outputs a similar compound name.

As illustrated in FIG. 1, the compound substitution device 10 includes an extraction unit 101, a frequency accumulation unit 102, and a score calculation unit 103. Furthermore, the compound substitution device 10 includes an analysis unit 104, a conversion unit 105, a superordinate concept search unit 106, a subordinate concept search unit 107, a selection unit 108, an inverse conversion unit 109, a substitution unit 110, a compound name generation unit 111, and a search unit 121. Furthermore, the compound substitution device 10 stores a knowledge graph 151, score information 152, a componentization rule 153, and a document database (DB) 154.

The knowledge graph 151 is a graph representing a relationship between a superordinate concept and a subordinate concept of a partial structure of a compound. For example, in the knowledge graph 151, there is a case where a plurality of subordinate concepts is associated with one superordinate concept.

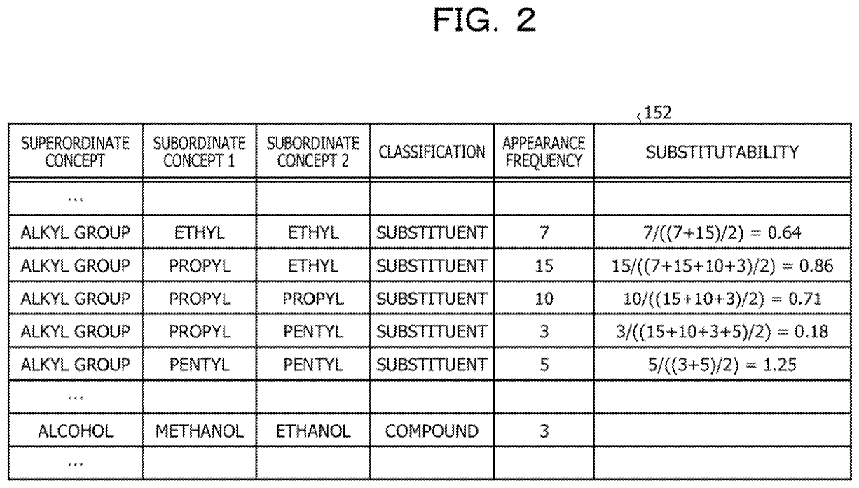

The score information 152 is information in which a combination of the superordinate concept and the subordinate concept before or after substitution is associated with a substitutability of each combination. FIG. 2 is a diagram illustrating an example of a data structure of score information. As illustrated in FIG. 2, a subordinate concept 1 that is a subordinate concept before being substituted and a subordinate concept 2 that is substituted subordinate concept are associated with a superordinate concept. Moreover, the score information 152 includes classification of the superordinate concept and the subordinate concept, an appearance frequency, and a substitutability. Note that, in the following description, the substitutability may be simply referred to as a score.

For example, FIG. 2 illustrates that classification of a combination of which the subordinate concept 1 is propyl, the subordinate concept 2 is ethyl is a substituent, an appearance frequency is 15, and a substitutability is 15/((7+15+10+3)/2)=0.86.

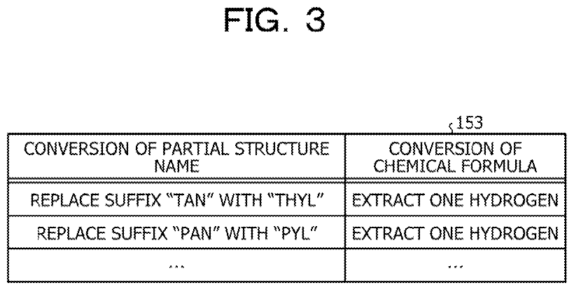

The componentization rule 153 is a rule for converting a partial structure of a compound into a substituent. FIG. 3 is a diagram illustrating an example of a data structure of a componentization rule. As illustrated in FIG. 3, the componentization rule 153 includes a conversion method of a partial structure name and a conversion method of a chemical formula. For example, FIG. 3 illustrates that, in a case where a partial structure name is converted by replacing a suffix “tan” with “thyl”, a chemical formula is converted by extracting one hydrogen.

The document DB 154 is a database that stores a document group. Documents stored in the document DB 154 are, for example, patent specifications, papers, books, or the like. The document may be included in a corpus to be described later that is stored in the document DB 154.

The extraction unit 101, the frequency accumulation unit 102, and the score calculation unit 103 generate the score information 152 based on documents in the field of chemistry. The documents are, for example, patent specifications, papers, books, or the like. Furthermore, a document used to generate the score information 152 is called a corpus.

The extraction unit 101 extracts information used to limit the superordinate concept and the subordinate concept from the corpus. The information extracted by the extraction unit 101 may be, for example, elements and the number of elements or may be a name of a structure or a chemical formula corresponding to the subordinate concept.

For example, it is assumed that the extraction unit 101 extract a ?.+ group of [element symbol][number][-˜][element symbol][number]. In this case, the extraction unit 101 extracts an element symbol “C” of the subordinate concept, extracts “1 to 4” as the number of the element symbols “C”, and extracts an “alkyl group” as the superordinate concept, from a sentence “R2 is a C1-C4 alkyl group that may include one or more fluorine atoms . . . ”.

Furthermore, for example, it is assumed that the extraction unit 101 extract ([partial structure],)+(or the like) as a.+ group. In this case, the extraction unit 101 extracts an “alkyl group” as the superordinate concept and extracts ethyl, propyl, and butyl as the subordinate concepts, from a sentence “an ethyl group, a propyl group, a butyl group, or the like can be exemplified as an alkyl group”.

The frequency accumulation unit 102 accumulates the information extracted by the extraction unit 101. First, the frequency accumulation unit 102 accumulates a condition included in the information extracted by the extraction unit 101 in a unified expression using the knowledge graph 151.

A procedure for accumulating the condition by the frequency accumulation unit 102 is as follows. For example, the frequency accumulation unit 102 searches the knowledge graph 151 for the superordinate concept. Next, when specifying a node of the superordinate concept, the frequency accumulation unit 102 traces nodes connected as the subordinate concepts in order, and acquires a rational formula by referring to a partial structure dictionary from a partial structure of each node. Moreover, the frequency accumulation unit 102 checks the acquired rational formula with the extracted condition.

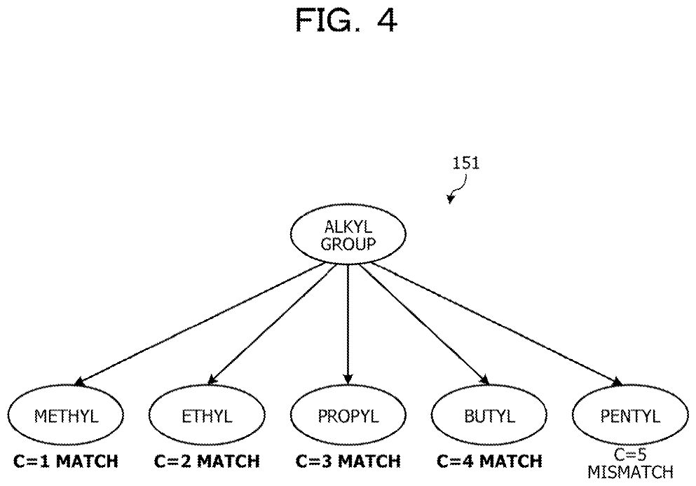

FIG. 4 is a diagram illustrating an example of a knowledge graph. Here, it is assumed that the superordinate concept included in the information extracted by the extraction unit 101 be an “alkyl group” and the condition be “the number of Cs is one to four”. At this time, as illustrates in FIG. 4, the frequency accumulation unit 102 specifies a node of the “alkyl group”. Then, the frequency accumulation unit 102 traces “methyl”, “ethyl”, “propyl”, “butyl”, and “pentyl” connected to the node of the “alkyl group” in order as the subordinate concepts, and obtains each rational formula. Of these, since the number of Cs of “methyl”, “ethyl”, “propyl”, and “butyl” is one to four, they meet the condition. On the other hand, since the number of Cs of “pentyl” is five, this does not meet the condition.

The frequency accumulation unit 102 increments an appearance frequency of a path from the subordinate concept to the subordinate concept, for the matched one. For example, the appearance frequency of the score information 152 is increased. Furthermore, in a case of a list of compound names, the frequency accumulation unit 102 increments appearance frequencies of the appeared subordinate concept and the combination of the superordinate concept and the subordinate concept.

The score calculation unit 103 calculates a substitutability (score) based on the appearance frequency of the score information 152. The score calculation unit 103 registers the calculated substitutability in the score information 152.

Here, it can be said that the extraction unit 101 extracts names of co-occurring partial structures. The score calculation unit 103 calculates the substitutability that is the score between the partial structures so as to be larger for a combination of partial structures that has a higher co-occurring probability based on a co-occurring frequency.

For example, since the substitutability is a probability that the superordinate concept is substituted with the subordinate concept, the score calculation unit 103 calculates a substitutability, for example, as indicated by the formula (1).

The substitutability between the subordinate concept 1 and the subordinate concept 2=an appearance frequency of a group of the superordinate concept and the subordinate concepts 1 and 2/(a sum of an appearance frequency of the subordinate concept 1 and an appearance frequency of the subordinate concept 2/2) (1)

Based on FIG. 2, a method for calculating a substitutability in a case where the superordinate concept is an “alkyl group”, the subordinate concept 1 is “propyl”, and the subordinate concept 2 is “ethyl” will be described. At first, it is assumed that the appearance frequency have been registered and the substitutability have not been registered.

First, an appearance frequency of a group of the superordinate concept and the subordinate concepts 1 and 2 is 15 as registered as the appearance frequency. Furthermore, since a sum of the appearance frequency of the subordinate concept 1 and the appearance frequency of the subordinate concept 2 is a sum of appearance frequencies in the row where “propyl” or “ethyl” appears as the subordinate concept 1 or 2, the sum is 7+15+10+3=35. As a result, the substitutability is 15/(35/2)=0.86.

The analysis unit 104, the conversion unit 105, the superordinate concept search unit 106, the subordinate concept search unit 107, the selection unit 108, the inverse conversion unit 109, the substitution unit 110, and the compound name generation unit 111 execute processing of outputting a similar compound name based on the compound name, by referring to the score information 152.

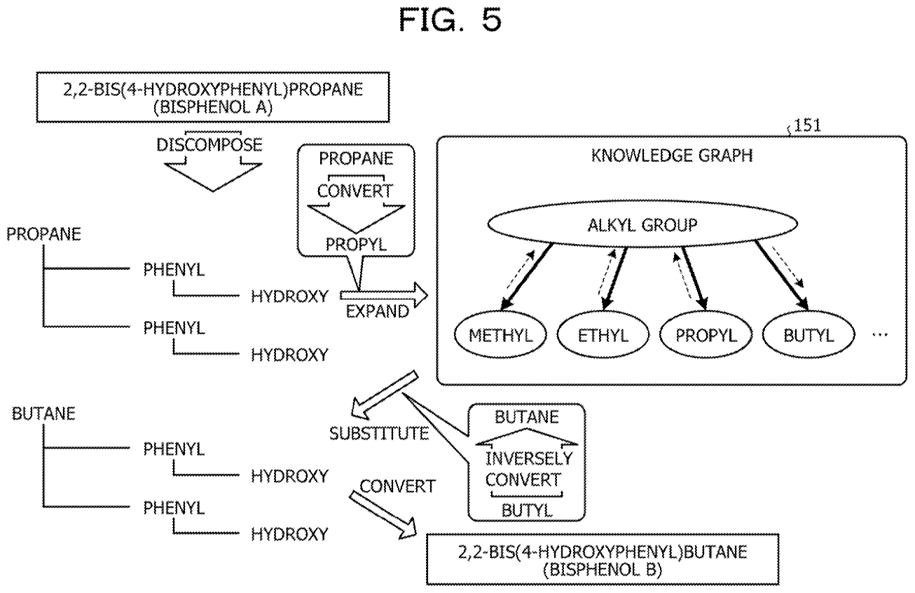

The analysis unit 104 analyzes the input compound name. For example, as illustrated in FIG. 5, the analysis unit 104 expands a compound indicated by the input compound name to a partial structure. FIG. 5 is a diagram for explaining processing of obtaining compounds having similar structures.

In the example in FIG. 5, the analysis unit 104 receives an input of a character string of “2,2-bis(4-hydroxyphenyl)propane”. 2,2-bis(4-hydroxyphenyl)propane is an example of a first compound.

The analysis unit 104 obtains a structure in which two phenyls are bonded to propane and hydroxy is further bonded to each phenyl, based on the character string of “2,2-bis(4-hydroxyphenyl)propane”. As illustrated in FIG. 5, the analysis unit 104 may represent a structure with tree-format data.

The conversion unit 105 specifies a first partial structure included in the first compound and converts a name of the specified first partial structure into a substituent name. The conversion unit 105 converts a name of a partial structure into a substituent name according to the componentization rule 153. For example, the conversion unit 105 can specify a partial structure that has an effect, as small as possible, on properties as the compound when being substituted with another partial structure, as the first partial structure. In the example in FIG. 5, the conversion unit 105 specifies propane as the first partial structure and converts the name “propane” into “propyl”.

The superordinate concept search unit 106 searches the knowledge graph 151 for the superordinate concept using the first partial structure as a key. Furthermore, the subordinate concept search unit 107 searches the knowledge graph 151 for the superordinate concept using the superordinate concept as a key.

The knowledge graph 151 in FIG. 4 indicates that methyl, ethyl, propyl, butyl, and pentyl exist as subordinate concepts of an alkyl group. For example, the knowledge graph in FIG. 4 indicates that the alkyl group exists as a common superordinate concept of methyl, ethyl, propyl, butyl, and pentyl.

For example, the superordinate concept search unit 106 searches the knowledge graph 151 using propyl as a key and obtains the alkyl group that is the superordinate concept. Then, the subordinate concept search unit 107 obtains methyl, ethyl, butyl, and pentyl, using the alkyl group that is the superordinate concept as a key. Note that a search result of the subordinate concept search unit 107 may include propyl that is the search key of the superordinate concept search unit 106.

The selection unit 108 refers to information indicating a relationship between a plurality of partial structures, and selects a second partial structure related to the first partial structure. The selection unit 108 selects a partial structure corresponding to the subordinate concept belonging to the same superordinate concept as the first partial structure as the second partial structure, based on a relationship between the superordinate concept and the subordinate concept between the partial structures, indicated in the information indicating the relationship between the plurality of partial structures. Furthermore, the selection unit 108 may select the plurality of partial structures as the second partial structures.

For example, the selection unit 108 selects some or all of the subordinate concepts searched by the subordinate concept search unit 107. The information indicating the relationship between the plurality of partial structures is, for example, a set of the subordinate concepts having the alkyl group as the superordinate concept in the knowledge graph 151, for example, methyl, ethyl, butyl, and pentyl.

The inverse conversion unit 109 inversely converts a name of the second partial structure selected by the selection unit 108 into a name of a partial structure. For example, the inverse conversion unit 109 inversely converts “methyl”, “ethyl”, “propyl”, “butyl”, and “pentyl” into “methane”, “ethane”, “propane”, “butane”, and “pentane”, respectively.

In a case where it is determined that the score is equal to or more than a threshold, the compound name generation unit 111 generates information indicating a second compound obtained by substituting the first partial structure of the first compound with the second partial structure. Furthermore, the substitution of the first partial structure with the second partial structure is performed by the substitution unit 110.

At this time, the compound name generation unit 111 generates the information indicating the second compound based on the second partial structure, selected by the selection unit 108 that satisfies conditions. For example, the compound name generation unit 111 generates the information indicating the second compound obtained by substituting the first partial structure of the first compound with a partial structure of which a score is determined to be equal to or more than the threshold, among the second partial structures.

The compound name generation unit 111 determines whether or not the score calculated based on an appearance status of a group including the first partial structure and the second partial structure in a plurality of pieces of text data is equal to or more than a threshold. Here, the score is the substitutability registered in the score information 152. The substitutability is an example of a score that increases as a frequency of appearance of the first partial structure and the second partial structure in the same piece of the text data included in the plurality of pieces of text data increases.

For example, it is assumed that the first compound be 2,2-bis(4-hydroxyphenyl)propane. Furthermore, it is assumed that the first partial structure be propyl. Furthermore, it is assumed that the selection unit 108 select methyl, ethyl, butyl, and pentyl as the second partial structures. Furthermore, it is assumed that the threshold of the substitutability be 0.6.

From FIG. 2, a substitutability in a case where propyl is substituted with ethyl is 0.86 and is equal to or more than the threshold. Therefore, the compound name generation unit 111 generates a name of a compound obtained by substituting propyl with ethyl. On the other hand, since a substitutability in a case where propyl is substituted with pentyl is 0.18 and is less than the threshold, the compound name generation unit 111 does not generate a name of a compound obtained by substituting propyl with pentyl. Furthermore, for example, if a substitutability is equal to or more than the threshold in a case where propyl is substituted with butyl, the compound name generation unit 111 generates “2,2-bis(4-hydroxyphenyl)butane” that is a name of a compound obtained by substituting propyl with butyl.

The search unit 121 receives the information indicating the first compound as an input and searches the document group stored in the document DB 154 for a document related to the information indicating the second compound generated by the compound name generation unit 111. For example, in a case where “2,2-bis(4-hydroxyphenyl)propane” is input to the compound substitution device 10 as a compound name, the search unit 121 can search for a document using “2,2-bis(4-hydroxyphenyl)butane” that is a similar compound name as a key. Note that the compound substitution device 10 may output the similar compound name or output the search result of the search unit 121.

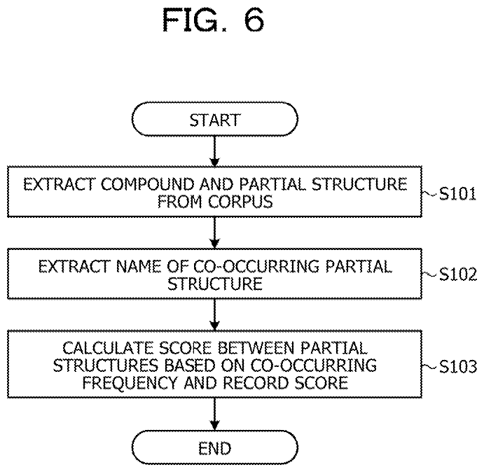

FIG. 6 is a flowchart illustrating a flow of processing of calculating a score. As illustrated in FIG. 6, first, the extraction unit 101 extracts a compound and a partial structure from the corpus (step S101) and extracts a name of a co-occurring partial structure (step S102). Then, the score calculation unit 103 calculates a score between the partial structures based on a co-occurring frequency and records the score in the score information 152. The co-occurring frequency is, for example, an appearance frequency in the score information 152.

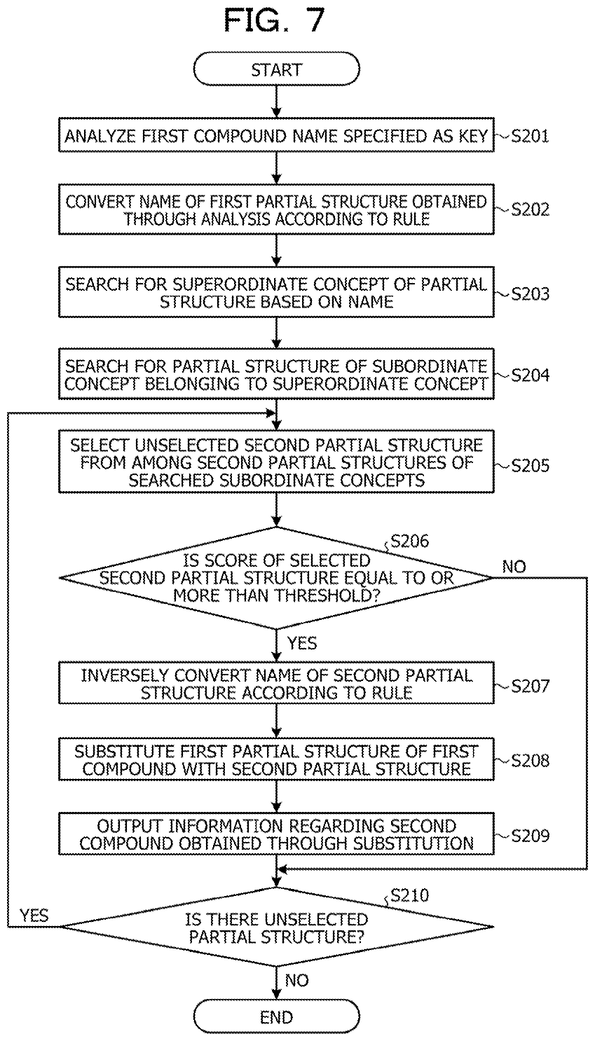

FIG. 7 is a flowchart illustrating a flow of processing of obtaining similar compounds. As illustrated in FIG. 7, first, the analysis unit 104 analyzes the first compound name specified as a key (step S201). Next, the conversion unit 105 converts a name of the first partial structure obtained through analysis according to a rule (step S202).

Here, the superordinate concept search unit 106 searches for a superordinate concept of the partial structure based on the name (step S203). Furthermore, the subordinate concept search unit 107 searches for a partial structure of a subordinate concept belonging to the superordinate concept (step S204). The superordinate concept search unit 106 and the subordinate concept search unit 107 search the knowledge graph 151.

The selection unit 108 selects an unselected second partial structure from among the second partial structures of the searched subordinate concepts (step S205). In a case where a score of the selected second partial structure is equal to or more than a threshold (step S206, Yes), the compound substitution device 10 proceeds to step S207. On the other hand, in a case where the score of the selected second partial structure is not equal to or more than the threshold (step S206, No), the compound substitution device 10 proceeds to step S210.

The inverse conversion unit 109 inversely converts a name of the second partial structure according to the rule (step S207). Then, the substitution unit 110 substitutes the first partial structure of the first compound with the second partial structure (step S208). Here, the compound name generation unit 111 outputs information regarding the second compound obtained through substitution (step S209). Furthermore, the compound substitution device 10 may search for a document using the information regarding the second compound as a key and output a search result.

In a case where there is an unselected partial structure (step S210, Yes), the compound substitution device 10 returns to step S205 and repeats the processing. Furthermore, in a case where there is no unselected partial structure (step S210, No), the compound substitution device 10 ends the processing.

As described above, the conversion unit 105 specifies the first partial structure included in the first compound. The selection unit 108 refers to information indicating a relationship between a plurality of partial structures, and selects a second partial structure related to the first partial structure. The compound name generation unit 111 determines whether or not the score calculated based on an appearance status of a group including the first partial structure and the second partial structure in a plurality of pieces of text data is equal to or more than a threshold. In a case where it is determined that the score is equal to or more than a threshold, the compound name generation unit 111 generates information indicating a second compound obtained by substituting the first partial structure of the first compound with the second partial structure. In this way, the compound substitution device 10 specifies a compound similar to the input compound, by considering the appearance status (for example, co-occurring frequency) of the group of the partial structures. Therefore, according to the present embodiment, it is possible to specify compounds having similar properties.

The selection unit 108 selects a partial structure corresponding to the subordinate concept belonging to the same superordinate concept as the first partial structure as the second partial structure, based on a relationship between the superordinate concept and the subordinate concept between the partial structures, indicated in the information indicating the relationship between the plurality of partial structures. The partial structure of the compound may belong to the superordinate concept such as an alkyl group or alcohol. Furthermore, the subordinate concepts belonging to the same superordinate concept may have similar properties. Therefore, according to the present embodiment, it is possible to specify the compounds having similar properties.

The search unit 121 receives the information indicating the first compound as an input and searches a document group for a document related to the information indicating the second compound generated by the compound name generation unit 111. As a result, a user can obtain a search result of a document regarding a compound similar to the compound only by inputting the information regarding the compound.

The compound name generation unit 111 determines whether or not the score that increases as the frequency of the appearance of the first partial structure and the second partial structure in the same piece of the text data included in the plurality of pieces of text data increases is equal to or more than the threshold. In this way, since compounds are more easily specified as similar compounds as the frequency of the appearance in the same document in actual is higher, according to the present embodiment, it is possible to improve accuracy for specifying the compounds having similar properties.

The selection unit 108 selects a plurality of partial structures corresponding to the subordinate concept belonging to the same superordinate concept as the first partial structure as the second partial structures, based on the relationship between the superordinate concept and the subordinate concept between the partial structures, indicated in the information indicating the relationship between the plurality of partial structures. The compound name generation unit 111 generates the information indicating the second compound obtained by substituting the first partial structure of the first compound with the partial structure, of which the score is determined to be equal to or more than the threshold, among the second partial structures. In this way, the compound substitution device 10 can obtain the similar compounds by substituting some partial structures. Therefore, according to the present embodiment, it is possible to efficiently specify compounds having similar properties.

The present embodiment is effective, for example, in a case where a document is searched using a compound name. In document search in the field of chemistry, there is a case where it is desired to consider a different notation (another name, chemical formula, SMILES, or the like) of a compound of which a name is input as a keyword and compounds that have similar structures or properties and do not have completely matching structures.

For example, if search can be performed as including a compound similar to the input compound as a key, this is effective in a case where a similarity between patent documents is determined. On the other hand, for example, in patent documents in the field of chemistry, there is a case where a large number of compounds are used in association with each other with a list of compound names, Markush claims, or the like, and it is considered to obtain a more useful search result by capturing these as a compound group at the time of the search. Furthermore, there is a case where an entire compound group is written in the Markush format in patent documents and only the small number of individual specific compound names are written. Moreover, in a case where search is performed using the compound name, to define a compound group including the compound name needs specialized knowledge, time, and labor. When any oversight occurs, this causes search omissions.

According to the present embodiment, for example, it is possible to obtain a name of a similar compound “2,2-bis(4-hydroxyphenyl)butane” with respect to an input of “2,2-bis(4-hydroxphenyl)propane”. At this time, a compound obtained by substituting with a partial structure with a lower co-occurring frequency is excluded. For example, in the example described above, 2,2-bis(4-hydroxyphenyl)pentane is excluded. As a result, according to the present embodiment, it is possible to obtain the name of the compound that can be used as a keyword used to obtain a more useful search result.

Pieces of information including a processing procedure, a control procedure, a specific name, various types of data, and parameters described above or illustrated in the drawings may be optionally changed unless otherwise specified. Furthermore, the specific examples, distributions, numerical values, and the like described in the embodiment are merely examples, and may be changed in any ways.

Furthermore, the respective components of the respective devices illustrated in the drawings are functionally conceptual, and the devices do not necessarily need to be physically configured as illustrated in the drawings. For example, specific forms of distribution and integration of each device are not limited to those illustrated in the drawings. For example, all or a part of the devices may be configured by being functionally or physically distributed or integrated in any units according to various types of loads, usage situations, or the like. Moreover, all or any part of individual processing functions performed in each device may be implemented by a central processing unit (CPU) and a program analyzed and executed by the CPU, or may be implemented as hardware by wired logic.

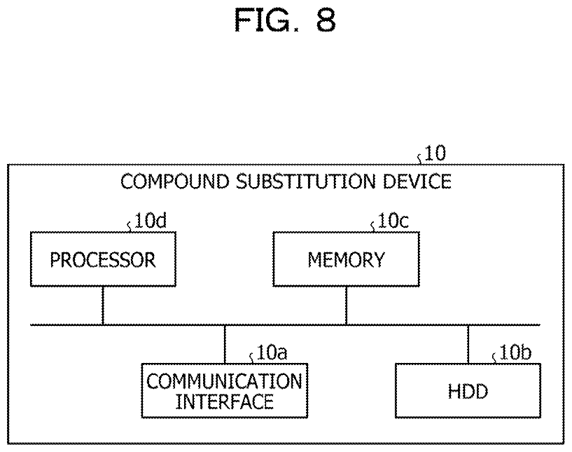

FIG. 8 is a diagram for explaining a hardware configuration example. As illustrated in FIG. 8, the compound substitution device 10 includes a communication interface 10a, a hard disk drive (HDD) 10b, a memory 10c, and a processor 10d. Furthermore, the individual units illustrated in FIG. 8 are connected to each other by a bus or the like.

The communication interface 10a is a network interface card or the like and communicates with another server. The HDD 10b stores a program that activates the functions illustrated in FIG. 1, and a DB.

The processor 10d is a hardware circuit that reads a program that executes processing similar to the processing of each processing unit illustrated in FIG. 1 from the HDD 10b or the like and loads the read program into the memory 10c, thereby operating a process that executes each function described with reference to FIG. 1 or the like. For example, this process executes functions similar to those of each processing unit included in the compound substitution device 10. For example, the processor 10d reads programs having similar functions to the conversion unit 105, the selection unit 108, the compound name generation unit 111, or the like from the HDD 10b or the like. Then, the processor 10d executes a process for executing processing similar to the conversion unit 105, the selection unit 108, the compound name generation unit 111, or the like.

As described above, the compound substitution device 10 operates as an information processing device that executes a compound substitution method by reading and executing a program. Furthermore, the compound substitution device 10 may implement functions similar to those of the embodiment described above by reading the program described above from a recording medium with a medium reading device and executing the read program described above. Note that other programs referred to in the embodiment are not limited to being executed by the compound substitution device 10. For example, the embodiment may be similarly applied to a case where another computer or server executes the program, or to a case where such computer and server cooperatively execute the program.

This program may be distributed via a network such as the Internet. Furthermore, this program may be recorded on a computer-readable recording medium such as a hard disk, flexible disk (FD), compact disc read only memory (CD-ROM), magneto-optical disk (MO), or digital versatile disc (DVD) and may be executed by being read from the recording medium by a computer.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a compound substitution program for causing a computer to execute processing comprising:
specifying a first partial structure included in a first compound;
referring to information that indicates a relationship between a plurality of partial structures and selecting a second partial structure related to the first partial structure;
determining whether or not a score calculated based on an appearance status of a group that includes the first partial structure and the second partial structure in a plurality of pieces of text data is equal to or more than a threshold; and
generating information that indicates a second compound obtained by substituting the first partial structure of the first compound with the second partial structure, in a case where it is determined that the score is equal to or more than the threshold.

2. The non-transitory computer-readable recording medium according to claim 1, wherein
the selecting processing includes processing of selecting a partial structure that corresponds to a subordinate concept that belongs to a same superordinate concept as the first partial structure as the second partial structure, based on a relationship between a superordinate concept and a subordinate concept between partial structures, indicated by the information that indicates the relationship between the plurality of partial structures.

3. The non-transitory computer-readable recording medium according to claim 1, for causing the computer to execute processing comprising:
receiving information that indicates the first compound as an input and extracting a document related to the information that indicates the second compound generated by the generating processing from a document group.

4. The non-transitory computer-readable recording medium according to claim 1, wherein
the score is a score that increases as a frequency of appearance of the first partial structure and the second partial structure in the same text data included in the plurality of pieces of text data increases.

5. The non-transitory computer-readable recording medium according to claim 1, wherein
the selecting processing includes processing of selecting a plurality of partial structures that corresponds to a subordinate concept that belongs to a same superordinate concept as the first partial structure as the second partial structure, based on a relationship between a superordinate concept and a subordinate concept between partial structures, indicated in the information that indicates the relationship between the plurality of partial structures, and
the generating processing includes processing of generating the information that indicates the second compound obtained by substituting the first partial structure of the first compound with a specific partial structure, among the plurality of partial structures, of which the score is determined to be equal to or more than the threshold.

6. A compound substitution method comprising:
specifying a first partial structure included in a first compound;
referring to information that indicates a relationship between a plurality of partial structures and selecting a second partial structure related to the first partial structure;
determining whether or not a score calculated based on an appearance status of a group that includes the first partial structure and the second partial structure in a plurality of pieces of text data is equal to or more than a threshold; and
generating information that indicates a second compound obtained by substituting the first partial structure of the first compound with the second partial structure, in a case where it is determined that the score is equal to or more than the threshold.

7. An information processing device comprising:
a memory; and
a processor coupled to the memory and configured to:
specify a first partial structure included in a first compound;
refer to information that indicates a relationship between a plurality of partial structures and selecting a second partial structure related to the first partial structure;
determine whether or not a score calculated based on an appearance status of a group that includes the first partial structure and the second partial structure in a plurality of pieces of text data is equal to or more than a threshold; and
generate information that indicates a second compound obtained by substituting the first partial structure of the first compound with the second partial structure, in a case where it is determined that the score is equal to or more than the threshold.
